(19) United States
(12) Patent Application Publication
(10) Pub. No.: US 2025/0025700 A1
(43) Pub. Date: Jan. 23, 2025

(54) REMOTE MONITORING OF CARDIAC IMD SIGNAL QUALITY

(71) Applicant: Cardiac Pacemakers, Inc., St. Paul, MN (US)

(72) Inventors: David Montilla Garcia, Badalona (ES); LeAnne M. Eberle, Mahtomedi, MN (US); Steven Donnelley, Lewiston, NY (US); Stefano Accinelli, Genova (IT)

(73) Assignee: Cardiac Pacemakers, Inc., St. Paul, MN (US)

(21) Appl. No.: 18/772,880

(22) Filed: Jul. 15, 2024

Related U.S. Application Data
(60) Provisional application No. 63/527,434, filed on Jul. 18, 2023.

(51) Int. Cl. A61N 1/37; A61N 1/372; G16H 40/40

(52) U.S. Cl. CPC A61N 1/37; A61N 1/37217; G16H 40/40

(57) ABSTRACT

Systems, devices and methods for determining sensing quality changes and drops in an implantable medical device, remotely and/or within the device itself. A cardiac implantable medical device tracks, over time, a number of counters, the contents of which are communicated to a processor which may be part of a remote monitoring device or which may be a customer service center that receives data from remote monitoring devices and/or programmers. Counter data is analyzed to identify changes in sensing quality and/or to provide information useful for clinical investigations, system integrity checking, or device reprogramming.

### CROSS REFERENCE TO RELATED APPLICATIONS

This application claims the benefit of U.S. Provisional Patent Application Ser. No. 63/527,434 filed Jul. 18, 2023, the disclosure of which is incorporated herein by reference.

### BACKGROUND

Implantable medical devices (IMDs) may include cardiac devices such as implantable pacemakers, defibrillators and implantable cardiac monitors. Such devices use a plurality of electrodes to sense cardiac signals which are then analyzed to make various determinations, including therapy decisions and determinations as to the need to store episode data. Some of these devices are capable of using multiple sensing configurations to sense cardiac signals, and so sensing configurations are typically set/selected by a physician during a programming session in the clinic and/or at implant. Over time, and/or in response to device faults, a previously selected sensing configuration may cease to be the optimal choice for a given patient and/or system. When that happens, a reconfiguration can be used to ensure that the IMD does not generate inappropriate therapy while continuing to be able to issue appropriate therapy when needed. New and/or alternative solutions that allow a device sensing configuration to be monitored and identified as in need of updating are desired.

### OVERVIEW

The present inventors have recognized, among other things, that a problem to be solved is the need for new and/or alternative multivariable analysis to identify patterns indicating possible oversensing and/or loss of signal quality in an early warning system.

A first illustrative and non-limiting example takes the form of a system for remotely monitoring a cardiac implantable medical device (IMD), the system comprising: a communication circuit configured to communicate wirelessly with a cardiac IMD in a remote monitoring (RM) session that generates RM session data; a processor configured to analyze RM session data to determine sensing quality for the IMD, wherein: the RM session data includes data for a plurality of counters present in the IMD; and the processor is configured to analyze RM session data to determine sensing quality for the IMD by: analyzing data in the plurality of counters; determining from the data in the plurality of counters that the IMD has experienced a drop in signal quality; and, if so, generating an alert indicating that the IMD has experienced a drop in signal quality.

Additionally or alternatively, the counter data does not include cardiac signal representations, such that the processor analyzes the RM session data to determine sensing quality without reviewing cardiac signal representations. Additionally or alternatively, the RM session data comprises data from a Tachy counter, the data in the Tachy counter indicating how frequently the IMD transitions between a first state in which cardiac rate is characterized as non-Tachycardia, and a second state in which the cardiac rate is characterized as Tachycardia or Fibrillation. Additionally or alternatively, the RM session data includes time data allowing the processor to determine a duration of time over which the Tachy counter has been used to count how frequently the IMD transitions between the first state and the second state. Additionally or alternatively, the processor and communication circuit are both contained in a remote monitoring device. Additionally or alternatively, the communication circuit is contained in a remote monitoring device which is communicatively coupled to a customer service center, and the processor is part of the customer service center. Additionally or alternatively, the remote monitoring device is configured to communicate to the IMD to take a corrective action in response to an alert.

Additionally or alternatively, the IMD is configured to analyze the cardiac signals and determine whether a sensing circuit of the IMD becomes saturated by the cardiac signals, and the IMD includes a saturation counter for counting how frequently the sensing circuit becomes saturated by the cardiac signals; the RM session data includes saturation counter data; and the processor is configured to analyze RM session data to determine sensing quality for the IMD by: determining whether the saturation counter data crosses a saturation counter data threshold and, if so, generating an alert indicating that the IMD has experienced a drop in signal quality.

Additionally or alternatively, the IMD is configured to analyze the cardiac signals to sense detected events, to analyze the detected events to identify noise, and includes a noise counter that counts how frequently detected events are identified as noise; the RM session data includes noise counter data; and the processor is configured to analyze RM session data to determine sensing quality for the IMD by: determining whether the noise counter data crosses a noise counter data threshold and, if so, generating an alert indicating that the IMD has experienced a drop in signal quality.

Additionally or alternatively, the IMD is configured to analyze the cardiac signals to sense detected events, to analyze the detected events to identify noise, and includes a persistent noise counter that counts how frequently sets of consecutive detected events are all identified as noise; the RM session data includes persistent noise counter data; and the processor is configured to analyze RM session data to determine sensing quality for the IMD by: determining whether the persistent noise counter data crosses a persistent noise counter data threshold and, if so, generating an alert indicating that the IMD has experienced a drop in signal quality.

Additionally or alternatively, the IMD is configured to analyze the cardiac signals to sense detected events, to analyze the detected events by comparison to a template, and includes a persistent mismatch counter that counts how frequently sets of consecutive detected events are all identified as not matching the template; the RM session data includes persistent mismatch counter data; and the processor is configured to analyze RM session data to determine sensing quality for the IMD by: determining whether the persistent mismatch counter data crosses a persistent mismatch counter data threshold and, if so, generating an alert indicating that the IMD has experienced a drop in signal quality.

Additionally or alternatively, the IMD is configured to analyze the cardiac signals to sense detected events, and to analyze the detected events to identify overdetection, and includes an overdetection counter that counts how frequently the IMD identifies overdetected events: the RM session data includes overdetection counter data; and the processor is configured to analyze RM session data to determine sensing quality for the IMD by: determining whether the overdetection counter data crosses a overdetection counter data threshold and, if so, generating an alert indicating that the IMD has experienced a drop in signal quality.

Additionally or alternatively, the system may further include the IMD, the IMD comprising a housing containing sensing circuitry, signal analysis circuitry, and therapy circuitry for issuing therapy signals; and a lead configured to couple to the IMD, such that the IMD takes the form of an implantable defibrillator. Additionally or alternatively, the IMD may be a subcutaneous-only implantable defibrillator.

Additionally or alternatively, the system may further include the IMD, wherein the IMD takes the form of an implantable cardiac monitor.

Another illustrative and non-limiting example takes the form of a method of monitoring operation of a cardiac implantable medical device (IMD), the method comprising: operating a communication circuit configured to communicate wirelessly with the IMD in a remote monitoring (RM) session that generates RM session data analyzing the RM session data to determine sensing quality for the IMD, wherein: the RM session data includes data for a plurality of counters present in the IMD; and the step of analyzing the RM session data to determine sensing quality for the IMD is performed by: analyzing data in the plurality of counters; determining from the data in the plurality of counters that the IMD has experienced a drop in signal quality; and, if so, generating an alert indicating that the IMD has experienced a drop in signal quality.

Additionally or alternatively, the counter data does not include cardiac signal representations, such that analysis of RM session data to determine sensing quality occurs without reviewing cardiac signal representations. Additionally or alternatively, the RM session data comprises data from a Tachy counter, the data in the Tachy counter indicating how frequently the IMD transitions between a first state in which cardiac rate is characterized as non-Tachycardia, and a second state in which the cardiac rate is characterized as Tachycardia or Fibrillation. Additionally or alternatively, the RM session data includes time data indicating a duration of time over which the Tachy counter has been used to count how frequently the IMD transitions between the first state and the second state, wherein the step of analyzing data includes accounting for the duration of time.

Additionally or alternatively, the step of analyzing data in the plurality of counters occurs in a processor of a remote monitoring device for the IMD, the remote monitoring device also including a communication circuit which also performs the communication step. Additionally or alternatively, the method may include the remote monitoring device instructing the IMD to take a corrective action in response to an alert to modify a setting or parameter in the IMD. Additionally or alternatively, the communication step is performed by a remote monitoring device which is communicatively coupled to a customer service center, and the analyzing step is performed at the customer service center. Additionally or alternatively, the method may include the remote monitoring device instructing the IMD to take a corrective action in response to an alert to modify a setting or parameter in the IMD. Additionally or alternatively, the IMD is configured to analyze the cardiac signals and determine whether a sensing circuit of the IMD becomes saturated by the cardiac signals, and the IMD includes a saturation counter for counting how frequently the sensing circuit becomes saturated by the cardiac signals; the RM session data includes saturation counter data; and the analyzing step is performed by: determining whether the saturation counter data crosses a saturation counter data threshold and, if so, generating an alert indicating that the IMD has experienced a drop in signal quality.

Additionally or alternatively, the IMD is configured to analyze the cardiac signals to sense detected events, to analyze the detected events to identify noise, and includes a noise counter that counts how frequently detected events are identified as noise; the RM session data includes noise counter data; and the analyzing step is performed by: determining whether the noise counter data crosses a noise counter data threshold and, if so, generating an alert indicating that the IMD has experienced a drop in signal quality.

Additionally or alternatively, the IMD is configured to analyze the cardiac signals to sense detected events, to analyze the detected events to identify noise, and includes a persistent noise counter that counts how frequently sets of consecutive detected events are all identified as noise; the RM session data includes persistent noise counter data; and the analyzing step is performed by: determining whether the persistent noise counter data crosses a persistent noise counter data threshold and, if so, generating an alert indicating that the IMD has experienced a drop in signal quality.

Additionally or alternatively, the IMD is configured to analyze the cardiac signals to sense detected events, to analyze the detected events by comparison to a template, and includes a persistent mismatch counter that counts how frequently sets of consecutive detected events are all identified as not matching the template; the RM session data includes persistent mismatch counter data; and the analyzing step is performed by: determining whether the persistent mismatch counter data crosses a persistent mismatch counter data threshold and, if so, generating an alert indicating that the IMD has experienced a drop in signal quality.

Additionally or alternatively, the IMD is configured to analyze the cardiac signals to sense detected events, and to analyze the detected events to identify overdetection, and includes an overdetection counter that counts how frequently the IMD identifies overdetected events: the RM session data includes overdetection counter data; and the analyzing step is performed by: determining whether the overdetection counter data crosses a overdetection counter data threshold and, if so, generating an alert indicating that the IMD has experienced a drop in signal quality.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an illustrative implantable medical device system;

FIG. 2 is a block diagram for an illustrative method of cardiac signal analysis;

FIG. 3 shows an illustrative example of a session file and counters;

FIG. 4 is a block diagram for an illustrative monitoring system;

FIGS. 5-7 show illustrative remote monitoring methods in block form; and

FIG. 8 shows an illustrative corrective action monitoring method.

### DETAILED DESCRIPTION

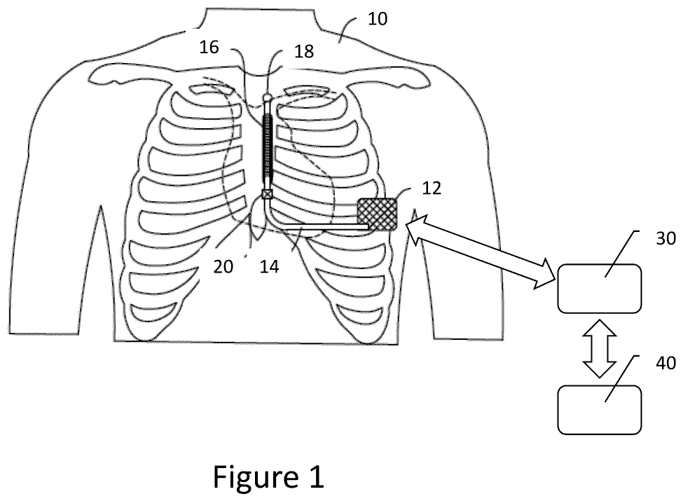

FIG. 1 shows an illustrative implantable medical device IMD system, in this case, a cardiac IMD system. The system is shown implanted in a patient 10. A pulse generator 12 is positioned near the left armpit, with a lead 14 extending toward the xiphoid process and then toward the head, over the sternum. A shock coil electrode 16 is shown between two sensing electrodes 18 and 20. For sensing the cardiac signal, the system is configured to use the conductive housing of the pulse generator 12 and the sense electrodes 18 and 20. For delivering a defibrillation shock in response to an identified ventricular tachyarrhythmia, the system will use the conductive housing of the pulse generator 12 and the shock coil electrode 16 as opposing poles. The IMD system as shown corresponds to the S-ICD System available from Boston Scientific which is described in various patents, such as U.S. Pat. Nos. 6,721,597, and 7,149,575, the disclosures of which are incorporated herein by reference.

As noted, three electrodes (18, 20, and the conductive housing of pulse generator 12) are available for sensing cardiac signals. A vector selection routine may be performed to identify a default sensing vector for cardiac signal analysis, using methods such as disclosed in U.S. Pat. Nos. 7,392,085 and/or 7,623,909, the disclosure of which is incorporated herein by reference. Vector selection may include multiple postural assessment to ensure that a sensing vector will work with the patient in different postures such as standing, seated and/or laying down, as described in U.S. Pat. No. 9,744,366, the disclosure of which is incorporated herein by reference. With a default sensing vector selected, cardiac signal analysis can be performed.

In other implementations, a cardiac signal monitor may take the place of the IMD system. For a signal monitor, the lead 14 is usually omitted, and a plurality of electrodes are positioned on the housing of the implantable device 12, which in the case of a monitor would house a microcontroller, memory, telemetry circuitry and a battery, and may omit output circuitry for generating therapy signals. If three electrodes are positioned on the monitor, sensing configurations may be set to choose a default sensing vector, if desired. When sensed signal exceed set thresholds, such as for rate, a monitoring device typically records cardiac signal data that can be later retrieved to assist in diagnosing the cardiac (disease) state for the patient 10.

External to the patient is a programmer 30, which is configured with wireless communications circuitry (using, for example, the Medradio communications band, inductive telemetry, and/or Bluetooth, or other suitable communications system) for communicating with the pulse generator 12. The programmer 30 can be used by a physician to obtain data from the pulse generator, such as cardiac signal data, device status data, and to program the pulse generator 12 with sensing and therapy instructions, as is well known in the art. The programmer may be a dedicated device, or may have other purposes. For example, a programmer 30 may take the form of a portable device, such as a cellphone/smartphone or tablet computer having a dedicated application (whether fully in control of a locked device, or operating as one of many applications on the device).

Device 30 may instead be a remote monitoring device. A remote monitor can be provided to the individual patient 10 and placed in the home, such as at the bedside. The remote monitor will periodically obtain data from the IMD system, including episode and/or status data, to generate session files which are further explained below. As with implementation of a programmer, a remote monitoring device may take the form of a cellphone/smartphone or tablet computer, if desired, running an application either as a locked, single purposes device, or having a user selectable application. For example, some medical device manufacturers have enabled or will soon enable patients to load applications to their smartphones or other devices to allow monitoring, and possibly of implantable devices. Device 30, regardless whether it is a programmer or a remote monitoring device, may also be configured to communicate data obtained from the IMD system to a customer service center 40 and/or to a remote server or cloud-based database/server 40 used by a maker or manager of the IMD system for monitoring and/or quality control purposes.

While FIG. 1 shows an S-ICD System with a subcutaneously positioned pulse generator 12 and lead 14, the current invention is not limited to this particular placement or system. A substernal system, in which the lead 14 would instead pass beneath the sternum, generally in the mediastinum, or a transvenous system, having a pulse generator 12 positioned more superior, such as near the clavicle, with a lead passing through one or more veins into the heart, as known in the art, may be used instead. Other positioning and configurations may be used as well. As long as the cardiac IMD is configured to sense cardiac signals using a sensing configuration and has the ability to transmit data from the IMD to an external device such as a programmer or remote monitor, the present invention may be implemented.

In addition to implementation as an IMD, the present invention may also be implemented in a wearable device, such as a wearable or external pacemaker or defibrillator. For example, the Lifevest® wearable defibrillator has sensing electrodes that are used to sense cardiac events and arrhythmias. An interrogation unit, such as a remote monitor or programmer, or an application operating on a general purpose device such as a cellphone or tablet computer, can obtain data from the wearable defibrillator, or the wearable defibrillator itself may be capable of communications using Broadband, WiFi or Cellular networks, to send session files to a remote server or database.

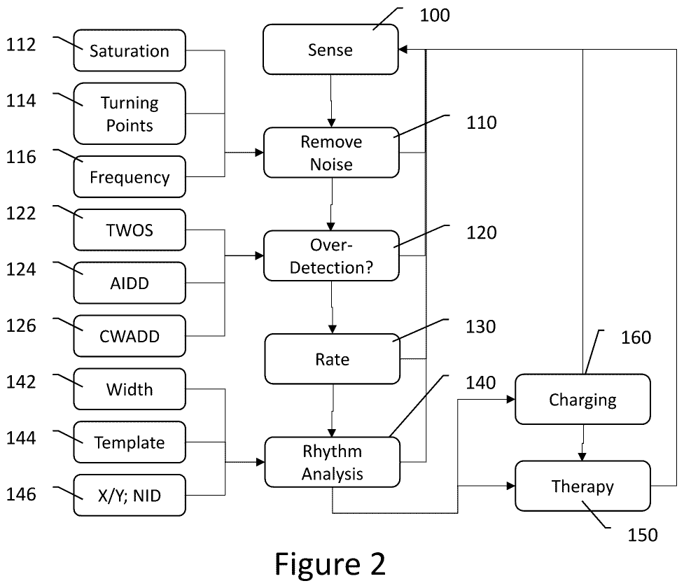

FIG. 2 is a block diagram for an illustrative method of cardiac signal analysis. The analysis may be performed by the implanted medical device on a microprocessor operating in accord with stored instruction sets, and/or one or more ASICs. Cardiac signals are sensed at 100, and are typically filtered, amplified, and digitized to allow signal analysis by a microcontroller and/or other operational circuitry (application specific integrated circuits, filters, capacitors, amplifiers, inductors, etc.). Typical parameters may include filtering to a bandpass of about 3 Hz to 40 Hz, with amplification allowing a signal in the range of up to several millivolts to be digitized. The sensed data is subject to event detection, in which, typically, the received cardiac signal is compared to one or more thresholds to detect signal elevation indicative of a cardiac event, such as the R-wave which indicates ventricular depolarization. Cardiac event detection may, for example, be performed as described in U.S. Pat. No. 9,802,056, the disclosure of which is incorporated herein by reference.

When a detected event occurs, data for the detected event is passed to a noise detection block 110, where detections caused by noise are identified and removed from analysis. Noise can be identified with any of several algorithms.

For example, saturation 112 may be identified if the cardiac signal is sufficiently large that it causes the amplifiers to reach a maximum output or “rail,” and/or if the average signal across a period of time exceeds a threshold. Saturation can be identified using methods disclosed in U.S. Pat. No. 8,831,711, and US PG Pat. Pub. 20100152799, the disclosures of which are incorporated herein by reference, though the present invention is not limited to those specific methods.

Noise may be identified by counting turning points 114 in a window of the cardiac signal surrounding the detected event, as disclosed in U.S. Pat. No. 7,248,921, the disclosure of which is incorporated herein by reference. Other analyses may be used instead for flagging noise in the detected events. Frequency content 116 may be relied upon instead, if desired, by, for example, comparing a signal content of the cardiac signal in a window to a threshold; if the threshold is exceeded, then the frequency content may be too high to represent an R-wave, and the detected event can be flagged as noise.

If a detected event is flagged as noise, the detected event can be rejected and the device returns to block 100. If noise is not identified at block 110, the method next moves to block 120, where the received detected event, along with on or more other detected events, can be analyzed to identify conditions indicating overdetection of the cardiac signal. From a high level, the noise removal at 110 may be understood as removing detected event that are non-cardiac in nature from the detected event queue, while overdetection 120 parses cardiac-source detected events to ensure that no more than one cardiac event is detected in each cardiac cycle. Some systems may treat events marked as overdetection and noise in the same way by discarding both. Illustrative examples of overdetection 120 may include T-Wave Overdetection (TWOS) 122, alternating interval double detection (AIDD) 124, and/or correlation waveform analysis double detection (CWADD) 126. Correlation analysis may also or instead include alternating event CWADD. Wide complex double detection analysis may also be used, as well as other width analysis. Some illustrative overdetection analyses are described in U.S. Pat. No. 8,160,686, the disclosure of which is incorporated herein by reference. Other methods may be used; for example, rather than using correlation waveform analysis, a device may use a

Wavelet transformation or principle component analysis to identify patterns of detected events that indicate overdetection and/or double detection.

Cardiac rate is then calculated as indicated at 130. Rate is often calculated by using the most recent intervals between non-noise and non-overdetected events, sometimes called R-R intervals, such as by using 4 R-R intervals to calculate rate. In many systems, one or more rate thresholds are used to classify the detected cardiac rate into one or more of normal sinus rhythm (NSR, which may not be normal or sinus originating but is typically, at least, not malignant), ventricular tachycardia (VT), or ventricular fibrillation (VF), with the last being the highest rate classification in some examples. If cardiac rate is classified as NSR, the system typically will return to sensing block 100 rather than proceeding with more detailed analysis, though that is not always the case.

If the rate 130 is above the NSR range, such as exceeding a VT or VF threshold, then rhythm analysis 140 takes place. Some systems will define a discrimination zone between VT and VF thresholds, such that if the rate is in the VF zone, a rate-only analysis will determine the rate to be malignant and if the rate is between VT and VF thresholds, further analysis takes place. Some examples of discrimination analysis may include comparing the sensed data for a particular detected event against thresholds for width 142, or against a static or dynamic template 144. An X/Y filter or number-of-intervals-to-detect (NID) counter 146 may be used as well. For example, an X/Y filter may test whether at least X out of the preceding Y non-noise and non-overdetected events have been classified as malignant; X/Y may be, for example and without limitation, 6/8, 12/16, 18/24, 24/32, or some other combination, which may be user selectable. An NID counter on the other hand looks at how many intervals between detected events indicate high (VT, such a 180 to 220 beats per minute) or very high (VF, such as 200 to 240 beats per minute) rates.

If rhythm analysis determines that a condition needing treatment is present, then either therapy or preparation for therapy may be called. For example, if rhythm analysis 150 identifies a condition that can be treated with anti-tachycardia pacing, such as a monomorphic VT, the device may proceed directly to therapy 150. If a condition necessitating defibrillation shock is identified, the device usually needs time to prepare by charging a high voltage capacitor or capacitor stack to a target energy/voltage, which may vary by device type/location (transvenous systems may defibrillate using up to 800 volts amplitude and 40 Joules of energy; an S-ICD on the other hand may use up to 1350 volts and 80 joules of energy). Charging block 160 is shown to this end; the analysis process may continue to iterate during charging, so that if the underlying cardiac rhythm returns to normal or non-malignant conditions during charging, therapy can be withheld, as discussed for example in U.S. Pat. Nos. 9,636,514 and 8,670,826 the disclosure of which is incorporated herein by reference. Once the process reaches a point where the rhythm analysis finds a malignant cardiac state is present and charging is complete, defibrillation shock can be delivered.

At several blocks in the analysis, the device may populate one or more counters. For example, the device may include counters that track how frequently noise is removed at block 110, as well as counters that track the reason for noise removal including a counter saturation 112, a counter for turning points 114 analysis being triggered/met, and/or to count the number of detected events that fail a frequency 116 analysis. Overdetection 120 may have its own counter, as well as counters that track the reason for any overdetection 120 flags, such as for TWOS 122, AIDD 124, CWADD 126, alternating CWADD, width analysis, or other overdetection analysis results.

Rate related counters can be provided as well, associated with block 130, including counters for how frequently rate crosses boundaries, such as from NSR to VT or VF ranges, or between VT and VF ranges (if both are defined). Such boundaries and the analysis involved, as well as any other changes in sensing/detection methods, may include hysteresis related rules. For example, if the system is in a first state in which the cardiac rate is characterized as NSR, or at least not malignant, when the detected cardiac rate (which may be determined by averaging the most recent one to eight, or four, for example, detected event intervals) exceeds a VT boundary, this may be deemed a Tachycardia or Tachyarrhythmia rate, and the device enters a second state which may be a Tachyarrhythmia state (note a further state change can occur later, when a treatable episode is declared, subject to additional criteria). Hysteresis can be implemented by requiring that the calculated rate drop below the VT boundary, or another boundary set at a lower rate or longer beat interval (such as the VT beat interval plus forty milliseconds, for example) than the VT boundary, for a predetermined period of time or quantity of consecutive iterations (such as four to fourty-eight, for example, twenty-four) of the analysis. Some examples may also transition to the NSR state upon delivery of therapy, if desired. The NSR-VT or NSR-Tachy counter, as it may be called, may increment only when all criteria have been met for the transition up (in an example, only rate needs to rise to enter the Tachy state), and again for the transition down (with hysteresis, in some examples). Each transition may increment the counter, or, optionally, only transitions going up, or down, may be counted, as desired. The use of this counter may parallel changes in how the received cardiac signal is analyzed to detect cardiac events. Changes to detection may including changing the detection profile used to identify potential cardiac signals, as disclosed in U.S. Pat. No. 9,802,056, the disclosure of which is incorporated herein by reference.

Rate related counters may indicate how frequently the NID or X/Y filter analyses are fulfilled, or reach an intermediate threshold (for example, if the VF NID is set to 12, a counter may be incremented anytime the VF NID reaches 9 so that “nearly VF” events can be tracked). The rhythm analysis block 140 may have additional counters, such as a counter that tracks how frequently a template matching analysis is met or fails. In an example, a counter may track how often a set number of consecutive events, or events within a time frame (such as a minute) fail to match a static template. For example, if a static NSR template is stored by the device, but matches less than half of detections over the course of a minute, the NSR template may be identified as not useful anymore, or it may be that something has changed with sensing (such as electrode migration, for example), and a counter can be used to track such issues. These counters and others can be used as further described below.

Another example counter may track identification of double detection after the device has calculated a rate in a tachycardia zone, that is, any zone that is not low rate or NSR. Such double detection flags may indicate that the device is calculating an elevated rate until such time as the tachycardia zone is reached. For example, some systems, as described in Cardiac event detection may, for example, be performed as described in U.S. Pat. No. 9,802,056, the disclosure of which is incorporated herein by reference, use an amplitude threshold against which the sensed cardiac signal is compared to detect events. The U.S. Pat. No. 9,802,056 patent describes an example in which the amplitude threshold is made more sensitive when an elevated rate is detected. Sporadic double detection may push the cardiac rate into an elevated zone without necessarily meeting all the rules applied to identify double detection as described in U.S. Pat. No. 8,160,686. Once the event detection threshold is lowered, the double detection may become more regular, and double detection rules will then be met more consistently. As the double detection algorithm works, the calculated cardiac rate will come back down, avoiding unnecessary episode declaration, capacitor charging, and/or inappropriate therapy. However, those corrective measures to eliminate the unnecessary charging operation may mask the sensing issues that are causing this up and down rate calculation. A counter that identifies double detection in an elevated rate zone may be useful to identify a problem with the underlying sensed signal.

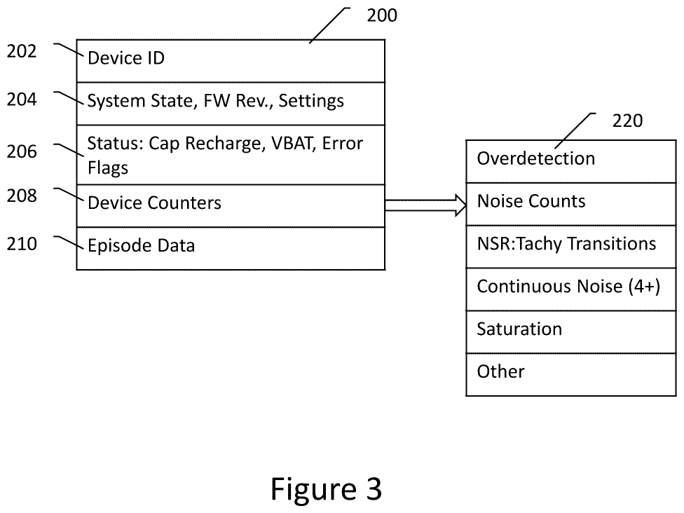

FIG. 3 shows an illustrative example of a session file and counters. As noted above, implantable devices (such as 12 in FIG. 1) may communicate to programmers and/or remote monitoring systems external to the patient. Communicated data may come in various packet formats, and the present invention is not intended to be limited to any particular form of data or data transmission. A typical session file is illustrated at 200 in FIG. 3. The session file 200 (sometimes called a logfile) may include device identification data at 202, such as a serial number. The session file 200 may also indicate current system state 204, which may also include firmware revision, and programmable parameters and other settings of the device, such as, without limitation, indicating thresholds used for VT and/or VF rate thresholds, therapy on/off parameters, amplitudes and waveform data, etc. Device status 206 may also be included, for example, reporting information about most recent capacitor recharge/reformation events, battery status such as battery voltage and/or expected battery life, and any error flags reportable from the device. Device counter data 208 may be provided as well, encompassing any or all of the counters previously described, or other counters, as may be suitable to a particular system.

Treated and untreated episode data may also be reported, as indicated at 210. As used herein, an “untreated” episode is an identified arrhythmia episode in which the system begins charging in preparation for a defibrillation therapy, but never delivers the therapy because the underlying sensed rhythm changes (whether due to actual change in the rhythm, due to removal of a noise source interfering with sensing, or because the device is able to resolve a sensing difficulty before therapy is issued). Episode data 210 may include cardiac signal representations, such as a series of voltage amplitude values sensed over the course of time during an identified episode of an arrhythmia. Episode data 210 may include treated and/or untreated episodes, if desired.

It should be noted that while episode data 210 includes cardiac signal representations, such as raw or compressed data, the device counters 208 will report only what events have been identified by the implantable medical device. That is, device counter data will not include actual representations of cardiac signal data. While a session file 200 may be representative of the data communicated by an IMD to a programmer during a clinical session, remote monitoring systems may limit the amount of data that is extracted to avoid unduly depleting the IMD battery, and to avoid prolonging telemetry sessions, among other issues. Thus, a session file 200 for a remote monitoring device may omit the episode data 210.

The device counters 208, in some examples, may include at least some of the counters referenced at 220. These may include overdetection counters. Overdetection counters may include any type of overdetection, or may be more granular to reflect specific types of overdetection. Noise counts may also be included in the list of counters, where each count represents a single noise event detected by the sensing circuitry and rejected by the noise rejection algorithm. NSR:Tachy Transitions may be counted as well, where NSR:Tachy indicates that the detected cardiac event rate has crossed the threshold from a low rate zone, which may be named NSR, to a high rate zone, which may be named Tachycardia. A continuous noise counter may be provided, where the counter is incremented in response to a series of consecutive noise markers occurring; in the example shown, at least four consecutive noise markers will increment the counter. In other examples, any string of 2+, 3+, 4+ or more consecutive noise markers may be used to increment the counter. A saturation counter may be included. Saturation may be determined using an average signal during a set period surrounding a detected event, a peak signal, or any other suitable measure that indicates either an amplitude that actually saturates input circuitry or which is of a type that saturation is either likely or in fact occurred. In an example, a signal which exceeds a peak threshold and does not return to zero within a predefined time frame may be considered saturated; other “saturation” definitions may be used.

Optionally other counters may be provided as desired. For example, a template mismatch counter may be provided, and may be incremented if, for example, a series of consecutive detected events fail to match a template. The template may be static (a stored representation of a normal beat, for example) or dynamic (based on recent in time detected events, such as a single preceding event or an average of a plurality of preceding detected events). One or more of these counters 220 may then be used to analyze cardiac signal data in the following examples.

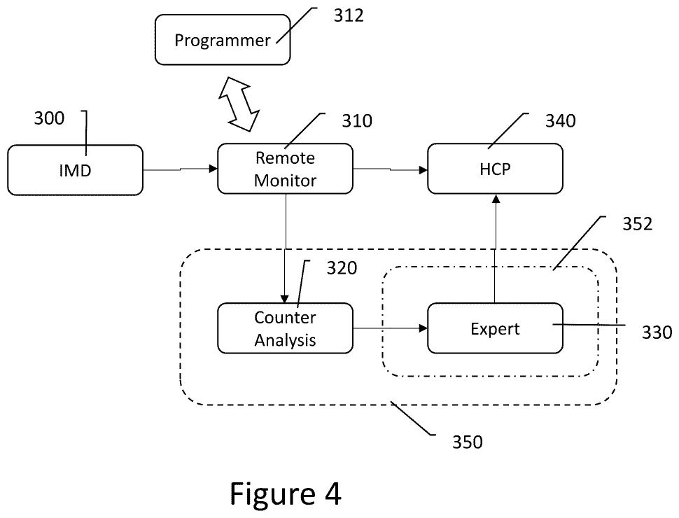

FIG. 4 is a block diagram for an illustrative monitoring system. An IMD 300 may be configured to communicate with a remote monitor 310, such as a bedside monitor, using wireless communications (such as but not limited to Medradio, inductive telemetry, and/or Bluetooth or other standard or non-standard wireless communication). Data from the remote monitor 310 may be reported directly to a healthcare provider (HCP) 340, as shown. Data from the remote monitor may instead or in addition be reported indirectly by passing to a customer service center 350 having a counter analysis processor 320 and an expert 330, which may be a human or an automated system. In another example, the customer service center may be as defined at 352, and the counter analysis processor 320 may be part of the remote monitor 310. In some examples, the HCP 340 may receive reports or other communications from the expert 330 and/or customer service center 350/352. As illustrated, a programmer 312 may be swapped into the system in the place of the remote monitor 310, if desired, and session data from the programmer used in place of and/or in addition to remote monitoring session data in the preceding and following examples.

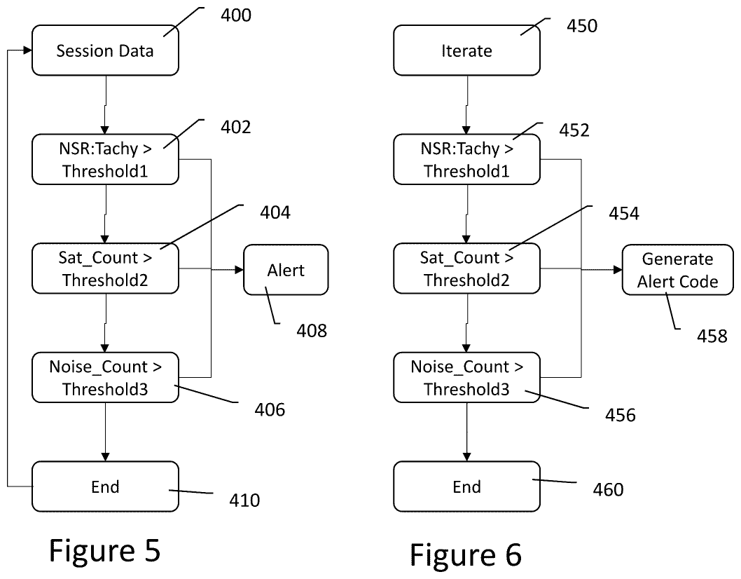

FIGS. 5-7 show illustrative remote monitoring methods in block form. Starting with FIG. 5, the process begins with receipt of session data at block 400, which may occur when the IMD is in proximity to a remote monitoring device and established, by wireless communications, a remote monitoring (RM) session, as may occur daily, weekly, biweekly or another interval. More frequent RM sessions deplete the battery more quickly but provide closer monitoring, as will be readily understood. As noted, the RM session data that is obtained by the remote device may include various counter data describe above, but typically would not include cardiac signal representations. In some alternative examples, cardiac signals representations may be included, such as to illustrate the contents of a morphology template, or as part of a treated or untreated episode data, as desired.

With new RM session data in, the NSR:Tachy counter is compared to a first threshold, which may be described as an NSR:Tachy threshold. The threshold may be an absolute threshold (such as one to five transitions per day, for example), or may be relative (such as by obtaining an average frequency of such transitions for a given patient/implant recipient). If the first threshold is exceeded, the method may generate an alert at 408 indicating a possible change or drop in device sensing signal quality. Such an alert would take place without the IMD declaring an episode of tachyarrhythmia in some examples. Using counter data, a minimum amount of data needs to be communicated, and no episode, whether treated or not, needs to take place. That is, some examples may perform the analysis without cardiac signal representations being communicated to the remote monitor, nor are cardiac signal representations analyzed by the remote monitor in some examples. The alert 408 can therefore be an early warning sign of issues in the patient/IMD.

An alert may be issued to bring an issue to the attention of an expert or customer service representative in some examples. An alert may instead or also be issued to bring the issue to the attention of a physician or HCP. In addition or instead of either of these, an alert may be issued to warn a user, such as by generating an alert on a remote monitoring device (which may be a smart phone in some examples), or issuing audible tones or sensory vibrations from an IMD or wearable device.

Additional comparisons can be made, by comparing the Saturation counter (Sat_Count) to a second threshold at 404, and/or a Noise_Count (which may be individual noise counters or consecutive noise counts) to a third threshold at 406. The system may generate the alert 408 if any one of the three tests 402, 404, 406 is met. In an alternative example, at least two of the three tests 402, 404, 406 would need to be met. In a still further example, all three tests 402, 404, 406 would need to be met. In other examples, only one, or only two of the three tests 402, 404, 406 may be included. Other counters, such as any of those described above and/or shown in FIG. 3, for example, may be compared to thresholds. Each threshold may be absolute or relative to the particular patient. If an alert is generated, the data may be passed on to an expert as shown in FIG. 4, for further assessment, and/or to the physician/HCP, or to the patient herself, as desired. The process in FIG. 5 may occur in a remote monitoring device, or may instead occur in a programmer used during a clinical visit/follow-up, if desired.

Counter data, and changes in counter data, can be analyzed from one session to another. A threshold analysis may include determining a difference from one session to the next for a given counter, and an alert may be generated based on such differences. For example, a threshold comparison may include the following inequalities:

$$Threshold > \vert Counter ( i ) - Counter ( i - 1 ) \vert$$

$$Or : Threshold > \vert Counter ( i ) - Counter\_Avg \vert$$

Where Counter_Avg is an average value for the given counter over time. Further statistical approaches can be used, for example, the threshold may be a standard deviation or variance (or multiple thereof), useful to identify a change or trend. Other analytics can be used as well.

When analyzing counter data from RM session files, received data may be smoothed to aid trend analysis and reduce the effect of any short-term anomalies in the data. For example, an alert detection may use a smoothing function such as:

Counter_Smoothed(i)=Counter(i)*A+Counter_Smoothed(i−1)*(1−A)

Where A is a smoothing factor or tuning factor in the range of [0:1]. The counter may be any of those discussed herein, for example, the NSR:Tachy counter can be assessed in the manner shown by the formula. For example, A may be any of 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, or some other intermediate value.

In some examples counter data may be normalized to account for time between RM session files being generated. For example, rather than analyzing counts alone, the analysis may be of counter per unit time, such as counts per day, counts per week, etc.

Furthermore, as noted, combinational elements may be considered as well. For example, multiple counters may be considered together. A quantity of NSR:Tachy transitions may be used to trigger assessment of a quantity of double detection identifications while in Tachy. Some examples may use the nature of the counter that causes an alert to determine a likely source of the issue. For example, cardiac oversensing, such as TWOS, is likely to trigger alerts of the NSR:Tachy type. Myopotential or muscle noise, endogenous to the patient, is more likely to create extended noise durations and/or NSR:Tachy type alerts, but not saturation events. Saturation events along with extended noise durations may be more likely caused by external or mechanical artifact noise, such as by lead fracture or displacement or proximity to a high voltage or high magnetic field.

The system alerts may be subject to tuning to more or less sensitive settings. If more sensitive settings are used, more alerts may be issued earlier, and this may generate alerts that do not actually require corrective actions. On the other hand, less sensitive settings may avoid false alarms, but may raise the likelihood of a patient receiving an inappropriate therapy or failing to receive needed therapy. Such tailoring of alert sensitivity may be left to the judgment of the physician. In some examples, tailoring of the settings for sensitivity may include determining how frequently RM session data is to be obtained and analyzed, with durations between monthly (less sensitive), bi-weekly (balanced) and weekly (more sensitive), for example.

As noted, instead of or in addition to RM session data, data and counters from a follow-up session with a patient using a programmer may be processed in the same manner as that shown in these Figures. That is, for example, when data from one session to the next is monitored and/or analyzed, counter data from a programmer-based follow-up, such as an in-person session in a clinic, may be used as one of the sessions, while RM session data is used for another of the sessions. This way, any counter data that is obtained in an in-person visit can be included in the analysis.

FIG. 6 shows another example operable in an IMD itself. Here the method starts at 450 with a new iteration. Iterations may be called periodically or in response to a specified event. For example, an IMD may perform the method as shown on a daily, weekly, bi-weekly, or other time-oriented bases. The IMD may instead perform the method in response to an event, such as a charging event, episode declaration, or therapy output.

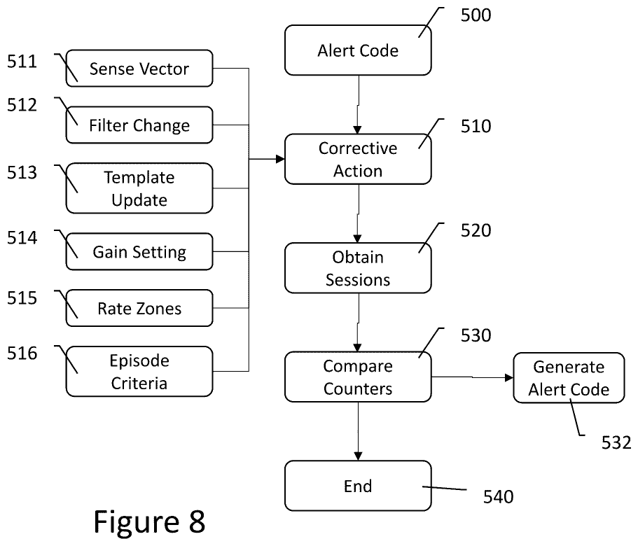

Once the new iteration is called at 450, the process is generally similar to that of FIG. 5, with comparison of various counters to corresponding thresholds as indicated at 452, 454 and 456. Meeting or crossing a threshold will result in an alert or alert code, as indicated at 458. Again, the alert 458 may be responsive to any one, two, or other specified combination of thresholds being met, as before. In addition to or as an alternative to the step of generating an alert 458, the IMD may instead take a corrective action such as modifying an operating parameter of the IMD, for example, any of the actions at 511-516 or otherwise described in relation to block 510 in FIG. 8, below. In an example, the new iteration may be called if the IMD has not had a follow-up session with a programmer, or a RM session with a remote monitor, for a period of time. For example, if the IMD determines that it has counter data, or episode data, which is to be overwritten (stored data can be overwritten in an IMD, for example in a first-in, first out order, or according to data priority rules, due to memory limitations), that may prompt the IMD to perform its own counter data analysis.

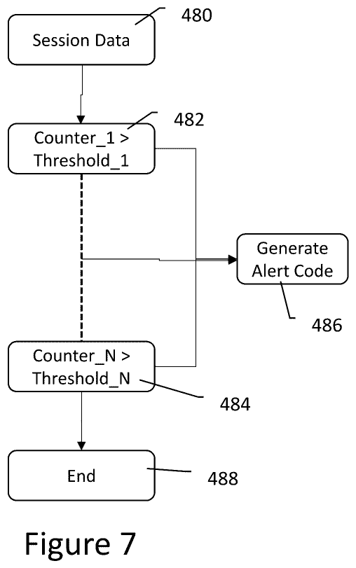

FIG. 7 illustrates a further example. Here, with RM session data received at 480, a plurality of counter-to-threshold comparisons are made as indicated at 482 to 484. When individual ones, combinations, or defined quantities of the thresholds are met, an alert code is generated. FIG. 7 may instead by operable by the IMD itself, as described with reference to FIG. 6, by triggering periodically or in response to prescribed events such as charging or therapy events.

As described above, a counter may be implemented to track identification of overdetection while the device is in an elevated rate zone. One of the comparisons at 482 to 484 may be specific to this particular counter, if desired.

In any of FIGS. 5-7, the alert or alert code may be communicated to the patient or to a physician/HCP, who could then be expected to schedule a follow-up with the patient and perform a further analysis of device status. For example, sensing reconfiguration may be triggered or performed, such a re-running vector selection and/or postural assessments, as described above. Any such corrective action may trigger use of the method shown in FIG. 8. An alert may instead be directed to the device manufacturer, a customer service representative or provider, or a device monitoring service provider, such as by generating a report, email, or other message.

In some examples, the alert code may prompt, or a corrective action may include, a request for the IMD to upload episode data including cardiac signal representations to the remote monitor for communication to a central server or to a physician. That is, while the standard RM session may omit the additional data, once the alert has been triggered, the costs associated with obtaining a larger amount of data from the IMD may be justified. As a result, additional data can be requested upon meeting the alert threshold, if desired.

FIG. 8 shows an illustrative corrective action monitoring method. Here, an alert code is generated at 500, leading to a corrective action at 510. Corrective actions may include modifying a sense vector, as indicated at 511, using an automated or manual process. For example, a manual sensing reconfiguration may be used to select a different sensing vector than that previously used even if automated sensing vector selection methods identify a vector that has triggered previous alerts at 500.

In another example, a filter change 512 may occur. For example, U.S. Pat. No. 10,149,627 describes a system that has a filter which can be turned on or off to enhance cardiac signal filtering by reducing the prevalence of signal data associated with T-waves, which are of a lower frequency content than R-waves in the cardiac signal. For example, a 9 Hz filter may be introduced or removed from the intake circuitry and/or input circuitry/analysis filters (a digital filter may be used, for example). Such a filter may be turned on if it was previously off in response to an alert code at 500, or may be turned off if it was previously on in response to an alert code at 500.

In another example, re-formation of a cardiac signal template may occur, as indicated at 513. Such a corrective action may, for example, be called for if the previous template did not match the sensed cardiac signal consistently and a counter associated with such template mismatch triggered the alert code at 500. In another example, a gain setting can be adjusted to modify, for example, how the incoming cardiac signal is amplified, as indicated at 514. For example, some systems have multiple levels of amplification available. Some systems may use an analog to digital converter (ADC) for digitizing received cardiac signal, and an adjustment may be made to the dynamic range of the ADC, if desired, which would effectively modify the gain setting as well.

In another example, one or more rate zone settings or boundaries can be modified, as indicated at 515. A rate zone boundary may be used to distinguish detected cardiac rates that are considered normal (or at least non-malignant), tachycardic, or fibrillation, for example. Boundaries may be, for example, adjusted up or down in response to an alert code at 500 as a corrective action 510. Arrhythmia episode criteria 516 can be adjusted if desired. For example, if an X/Y filter is used (in which X cardiac events of Y preceding events need to be deemed arrhythmic to trigger an episode and potential therapy), any of X, Y, or both X and Y may be adjusted up or down. Still further, some systems have episode criteria requiring some degree of persistence before an arrhythmia episode is declared, such as having 2, 5, 8 or more consecutive iterations of analysis meeting episode declaration criteria; increasing the number of consecutive iterations required may be another corrective action. In some systems, an NID (number of indications to detect) arrhythmia detector is used; the NID value (typically in the range of 8 to 24, or lower or higher) may be adjusted as a corrective action. Other modifications to the manner in which cardiac signals are sensed, filtered and/or analysis to make arrhythmia determinations can be deemed corrective actions 510 as well.

Corrective action 510 may instead include surgical intervention to anchor an electrode or lead in a desired position if it is determined that the electrode or lead has been displaced or migrated out of a desired position.

A corrective action 510 may take place with the assistance of a physician or medical personnel interacting with the patient having the IMD during a clinical follow-up session. For example, the programmer 30 (FIG. 1) may be used to modify any of the device settings or parameters noted at 511-516 during such a clinical follow-up session. Alternatively, a corrective action 510 may occur in a system as shown in FIG. 4 by having the remote monitor 310 communicate, using instructions sent by the customer service center 350, new system parameters or settings to use for the IMD 300.

After any of these, or other corrective actions occur, RM session data is obtained as indicated at 520. Counters can then be compared to thresholds, as indicated at 530, which may entail performing the methods of any of FIG. 5-7, for example. If the situation has improved as indicated by the counter values being reduced, meaning that the various indicators of possible sensing difficulties are not being triggered as frequently, the method ends at 540. If, on the other hand, sensing difficulties are increasing or have not changed, a new alert code is generated as indicated at 532.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein. In the event of inconsistent usages between this document and any document incorporated by reference, the usage in this document controls. In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of monitoring operation of a cardiac implantable medical device (IMD), the method comprising:
operating a communication circuit configured to communicate wirelessly with the IMD in a remote monitoring (RM) session that generates RM session data;
analyzing the RM session data to determine sensing quality for the IMD, wherein:
the RM session data includes data for a plurality of counters present in the IMD; and
the step of analyzing the RM session data to determine sensing quality for the IMD is performed by:
analyzing data in the plurality of counters;
determining from the data in the plurality of counters that the IMD has experienced a drop in signal quality; and, if so,
generating an alert indicating that the IMD has experienced a drop in signal quality.

2. The method of claim 1, wherein the counter data does not include cardiac signal representations, such that analysis of RM session data to determine sensing quality occurs without reviewing cardiac signal representations.

3. The method of claim 1, wherein the RM session data comprises data from a Tachy counter, the data in the Tachy counter indicating how frequently the IMD transitions between a first state in which cardiac rate is characterized as non-Tachycardia, and a second state in which the cardiac rate is characterized as Tachycardia or Fibrillation.

4. The method of claim 1, wherein the step of analyzing data in the plurality of counters occurs in a processor of a remote monitoring device for the IMD, the remote monitoring device also including a communication circuit which also performs the communication step.

5. The method of claim 4, further comprising the remote monitoring device instructing the IMD to take a corrective action in response to an alert to modify a setting or parameter in the IMD.

6. The method of claim 1, wherein the communication step is performed by a remote monitoring device which is communicatively coupled to a customer service center, and the analyzing step is performed at the customer service center.

7. The method of claim 6, further comprising the remote monitoring device instructing the IMD to take a corrective action in response to an alert to modify a setting or parameter in the IMD.

8. The method of claim 1, wherein:
the IMD is configured to analyze the cardiac signals and determine whether a sensing circuit of the IMD becomes saturated by the cardiac signals, and the IMD includes a saturation counter for counting how frequently the sensing circuit becomes saturated by the cardiac signals;
the RM session data includes saturation counter data; and
the analyzing step is performed by:
determining whether the saturation counter data crosses a saturation counter data threshold and, if so,
generating an alert indicating that the IMD has experienced a drop in signal quality.

9. The method of claim 1, wherein:
the IMD is configured to analyze the cardiac signals to sense detected events, to analyze the detected events to identify noise, and includes a noise counter that counts how frequently detected events are identified as noise;
the RM session data includes noise counter data; and
the analyzing step is performed by:
determining whether the noise counter data crosses a noise counter data threshold and, if so,
generating an alert indicating that the IMD has experienced a drop in signal quality.

10. The method of claim 1, wherein:
the IMD is configured to analyze the cardiac signals to sense detected events, to analyze the detected events to identify noise, and includes a persistent noise counter that counts how frequently sets of consecutive detected events are all identified as noise;
the RM session data includes persistent noise counter data; and
the analyzing step is performed by:
determining whether the persistent noise counter data crosses a persistent noise counter data threshold and, if so,
generating an alert indicating that the IMD has experienced a drop in signal quality.

11. The method of claim 1, wherein:
the IMD is configured to analyze the cardiac signals to sense detected events, to analyze the detected events by comparison to a template, and includes a persistent mismatch counter that counts how frequently sets of consecutive detected events are all identified as not matching the template;
the RM session data includes persistent mismatch counter data; and
the analyzing step is performed by:
determining whether the persistent mismatch counter data crosses a persistent mismatch counter data threshold and, if so,
generating an alert indicating that the IMD has experienced a drop in signal quality.

12. The method of claim 1, wherein:
the IMD is configured to analyze the cardiac signals to sense detected events, and to analyze the detected events to identify overdetection, and includes an overdetection counter that counts how frequently the IMD identifies overdetected events:
the RM session data includes overdetection counter data; and
the analyzing step is performed by:
determining whether the overdetection counter data crosses a overdetection counter data threshold and, if so,
generating an alert indicating that the IMD has experienced a drop in signal quality.

13. A system for remotely monitoring a cardiac implantable medical device (IMD), the system comprising:
a communication circuit configured to communicate wirelessly with a cardiac IMD in a remote monitoring (RM) session that generates RM session data;
a processor configured to analyze RM session data to determine sensing quality for the IMD, wherein:
the RM session data includes data for a plurality of counters present in the IMD; and
the processor is configured to analyze RM session data to determine sensing quality for the IMD by:
analyzing data in the plurality of counters;
determining from the data in the plurality of counters that the IMD has experienced a drop in signal quality; and, if so,
generating an alert indicating that the IMD has experienced a drop in signal quality.

14. The system of claim 13, wherein the counter data does not include cardiac signal representations, such that the processor analyzes the RM session data to determine sensing quality without reviewing cardiac signal representations.

15. The system of claim 13, wherein the RM session data comprises data from a Tachy counter, the data in the Tachy counter indicating how frequently the IMD transitions between a first state in which cardiac rate is characterized as non-Tachycardia, and a second state in which the cardiac rate is characterized as Tachycardia or Fibrillation.

16. The system of claim 13, wherein the processor and communication circuit are both contained in a remote monitoring device.

17. The system of claim 13, wherein the communication circuit is contained in a remote monitoring device which is communicatively coupled to a customer service center, and the processor is part of the customer service center.

18. The system of claim 17, wherein the remote monitoring device is configured to communicate to the IMD to take a corrective action in response to an alert.

19. A method of monitoring operation of a cardiac implantable medical device (IMD), the IMD maintaining a plurality of counters for events that the IMD identifies, the method comprising:
analyzing data in the plurality of counters;
determining from the data in the plurality of counters that the IMD has experienced a drop in signal quality; and, if so,
generating an alert indicating that the IMD has experienced a drop in signal quality or modifying an operating parameter of the IMD.

20. The method of claim 19, wherein the plurality of counters includes a Tachy counter, the data in the Tachy counter indicating how frequently the IMD transitions between a first state in which cardiac rate is characterized as non-Tachycardia, and a second state in which the cardiac rate is characterized as Tachycardia or Fibrillation, and the determining step is performed by comparing the data in the Tachy counter to one or more thresholds.
